(19) United States
(12) Patent Application Publication
(10) Pub. No.: US 2008/0302371 A1
(43) Pub. Date: Dec. 11, 2008

(54) Boot for Ulcer Treatment

(76) Inventors: Orly Cohen, Franklin, TN (US); William A. Cohen, Franklin, TN (US); Michael Baglione, Fairview, TN (US)

Correspondence Address: WADDEY & PATTERSON, P.C., 1600 DIVISION STREET, SUITE 500, NASHVILLE, TN, 37203, US

(21) Appl. No.: 11/758,551

(22) Filed: Jun. 5, 2007

(51) Int. Cl. A61F 13/06

(52) U.S. Cl. 128/888; 36/87

(57) ABSTRACT

A boot for the treatment of an ulcer wherein the boot is comprised of at least 80% by weight of closed cell polyethylene. The boot comprises a single article and covers a foot and lower part of the leg of a person. An interior surface of the boot is vacuum and heat molded to the foot and lower leg of the person, and a cut out is formed to surround the ulcer of the person. This off loads the weight of the person from the site of the ulcer, and the custom molding minimizes any slipping and shearing forces on the ulcer. The boot is lightweight, easy to put on and take off, and less expensive than other healing boots available.

### BACKGROUND OF THE INVENTION

A. Field of the Invention

The current invention relates to a boot to aid in the treatment of ulcers on the foot, ankle, and/or lower leg of a patient. The boot offloads the weight from the ulcer and transfers the weight to the rest of the foot, minimizing direct pressure and shear forces on the ulcer.

B. Description of the Related Arts

People or patients with diabetes mellitus and other medical conditions i.e. status post chemotherapy, idiopathic, alcoholic, and/or neuromuscular conditions, frequently develop neuropathy, or a loss of feeling, particularly in the lower extremities such as the foot or lower leg. Due to this neuropathy, they do not feel insipient injuries, which tend to develop into more serious injuries because the individual does not adjust to minimize the pain and trauma. The pain people without neuropathy feel cause them to make adjustments which serves to minimize the pain, and therefore further trauma to the condition is also minimized. The adjustments can be as simple as limping or shifting the weight from one foot to the other. People with diabetes mellitus tend to get ulcers on the plantar surface and sometimes on other surfaces of the foot. It is common for people with diabetic neuropathy to suffer amputation of the foot and lower leg as a result of complications from diabetic ulcers.”

Once an ulcer has developed, diabetic ulcer treatment involves off loading the ulcer and reducing the direct pressure and shear forces at the sight of the ulcer. The patients have a reduced sensation in the foot, so they often don't have the pain involved with their ulcer. The patients have a reduced feeling in the foot, so they often don't notice the pain involved with their ulcer. Because of this, patient adherence to a treatment regime can be spotty, especially if the treatment regime is inconvenient. Clearly, ease and convenience of the regime will increase patient compliance resulting in improved efficacy. Some treatments which have been used include bed restrictions, which is very inconvenient for the patient. Crutches or wheel chairs have also been utilized, which is less inconvenient than bed restriction, but is still very inconvenient for the patient. Casts have been used, especially a total contact cast, to off weight the ulcer. Although this treatment can be effective, it is complex and time consuming. The cast has to be changed frequently, and the application of the cast must be done by a skilled person.

There are many specialized boots to aid in the treatment of ulcers, especially the ulcers typically associated with diabetes mellitus. To maximize the effectiveness of these boots, they should be easy to put on and take off, relatively inexpensive, convenient to use, and capable of being adapted to changing foot and leg sizes of the patient. One example of such a boot is described in U.S. Pat. No. 6,945,946 by Rooney. This invention includes a custom made orthosis which has a rigid L shaped support member with a rigid anterior support shell hingedly connected to the L shaped support member. There is a hollow on the bottom or sole of the L shaped support member, and this hollow is positioned to be adjacent to the ulcer of one particular patient. There are securing straps which are used to attach the anterior support shell to the L shaped support member.

Jensen, et al describes another boot in U.S. Pat. No. 6,682,497. This boot includes a pair of rigid shells joined together with a fastening system to form a unified brace. A combination of bladders within the rigid shells engage the foot and lower leg of the person and serve to off weight the bottom or plantar surface of the foot. The volume of at least one of the bladders is adjustable so as to maintain a uniform pressure on the person's foot and lower leg. The pressure and size of the brace can be adjusted by the amount of fill included in the bladders.

Another boot is described by Brady in U.S. Pat. No. 5,197,942. Brady describes a foot orthosis which has a rigid brace with a back and a sole portion. The brace has a front portion which can be removed from the back and sole portion, and straps connect the two portions of the brace. The straps serve to fasten the orthosis securely to the foot and lower leg of the patient. This boot also includes an aperture in the sole portion positioned to be adjacent to the ulcer of the patient.

U.S. patent application No. 2003/0196352, by Bledsoe, et al, describes an orthopedic walking boot. This orthopedic walking boot has a hard, unyielding shell designed for walking. The shell supports a midsole with a foot shaped bed. The midsole has a foot shaped cavity with rounded sides to support the heel, arch, sides, and bottom of the patient's foot. An inner sole fits over the foot shaped cavity, and is compressed in response to foot pressure both on the sides and the bottom of the foot. The shell, midsole and inner sole, serve to transfer some weight from the bottom of the foot to the sides and arch of the foot, thereby lowering the peak pressure on the bottom surface of the foot. A breathable booty wraps around the lower leg and the foot for added protection.

Jereome, et al, describes another boot in U.S. patent application No. 2006/0135899. This boot has a shell with a hinge located at the end of the toe portion of the boot. The shell has a lower part and an upper part, wherein the upper part overlaps the lower part when the shell is closed. Straps are used to secure the boot in the closed position. There is an insole which is customized to fit at least part of the bottom and sides of a particular patient's foot. The insole can include recesses positioned to be adjacent to the ulcer on the patient's foot.

### BRIEF SUMMARY OF THE INVENTION

The current invention comprises a boot formed primarily of a closed cell polyethylene which extends up at least part of a leg of a person or patient. The boot has an interior surface defined by an upper and an insole which is custom molded to the entire foot and part of the lower leg. There is a cut out on the inner surface of the boot, and the cut out is positioned to surround an ulcer. The cut out serves to off weight and aerate the ulcer, which aids the healing process. There is an expansion slot defined in the upper, and a flap to close the slot. The flap is sealed in the close position with a releasable fastener such as a hook and loop fastener. The boot includes a rocker shaped outsole for ground engagement. The rocker shape facilitates the gate, helps to minimize shear forces, and reduces pressure on the ulcer.

### BRIEF DESCRIPTION OF THE SEVERAL VIEWS OF THE DRAWINGS

FIG. 1 is a front view of the boot with the flap closed over the outer flap.

FIG. 2 is a front view of the boot with the flap and expansion slot opened.

FIG. 3 is a front view of the boot with no flap and a strap to compress the expansion slot.

FIG. 4 is a side view of the boot with the flap and expansion slot opened.

FIG. 5 is a top view of the boot with the flap closed over the expansion slot.

### DETAILED DESCRIPTION OF THE INVENTION

### Upper

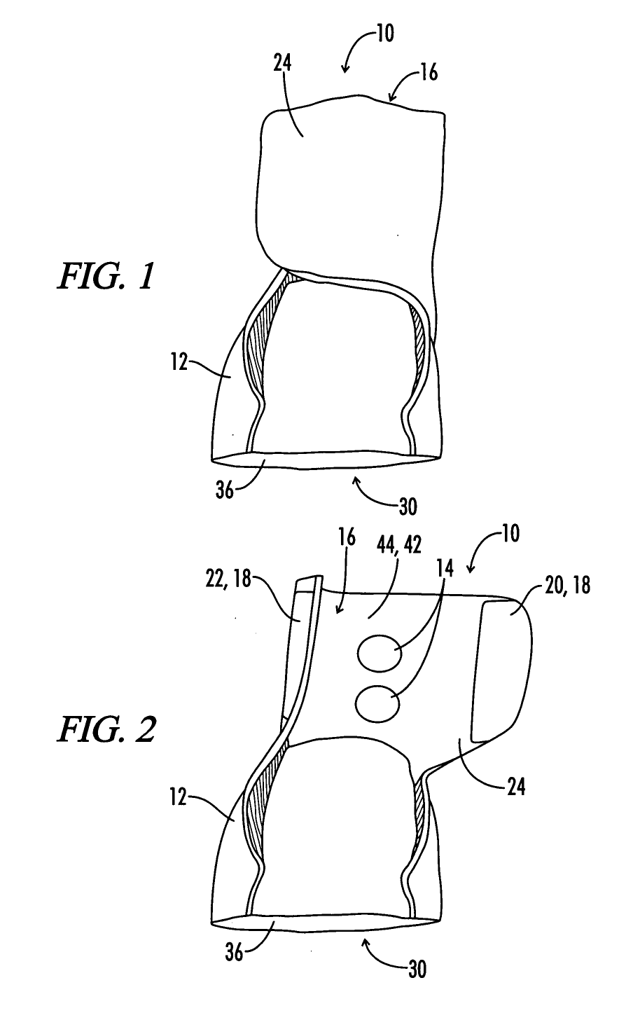

The current invention comprises a boot 10 having an upper 12, as seen in FIGS. 1 and 2. The upper 12 is dimensioned to extend up at least part of a leg of a person, but only to a point below the knee. Different versions of the boot 10 can extend to different heights on the leg of the wearer. The upper 12 is somewhat flexible, which allows for some flexion of the ankle joint when the person walks or ambulates. Ventilation holes 14 can be defined in various locations in the upper 12 to help cool the leg and foot. However, it is also contemplated that the upper 12 be solid to more completely enclose the foot and leg, and to better protect the patient.

The upper 12 also includes an expansion slot 16 to facilitate the donning and removable of the boot 10. The expansion slot 16 is comparable to the opening in most shoes which is filled by the tongue. In the current invention, the expansion slot 16 can be collapsed or compressed by a releasable fastener 18 to secure the boot 10 in the closed position, The releasable fastener 18 is connected to the upper 12, and is positioned to overlap and compress the expansion slot 16 so as to close the boot 10 around the person wearing the boot 10.

In one embodiment of the invention, the releasable fastener 18 comprises a hook and loop fastener. The releasable hook and loop fastener 18 includes a hook patch 20 and a loop patch 22; however, there are many types of releasable fasteners 18 which could be used. The hook patch 20 is depicted on a flap 24 whereas the loop patch 22 is shown on the upper 12 adjacent the expansion slot 16. The flap 24 is defined on the upper 12, and is positioned adjacent to the expansion slot 16. The flap 24 is laid over the expansion slot 16 and connected to the upper 12 to compress the expansion slot 16 and close the boot 10. When the boot 10 is closed, the flap 24 overlaps the expansion slot 16, and is releasably secured to the upper. The hook patch 20 and the loop patch 22 could be reversed, so the loop patch 22 was on the flap, as long as the hook patch 20 and the loop patch 22 are positioned to connect together.

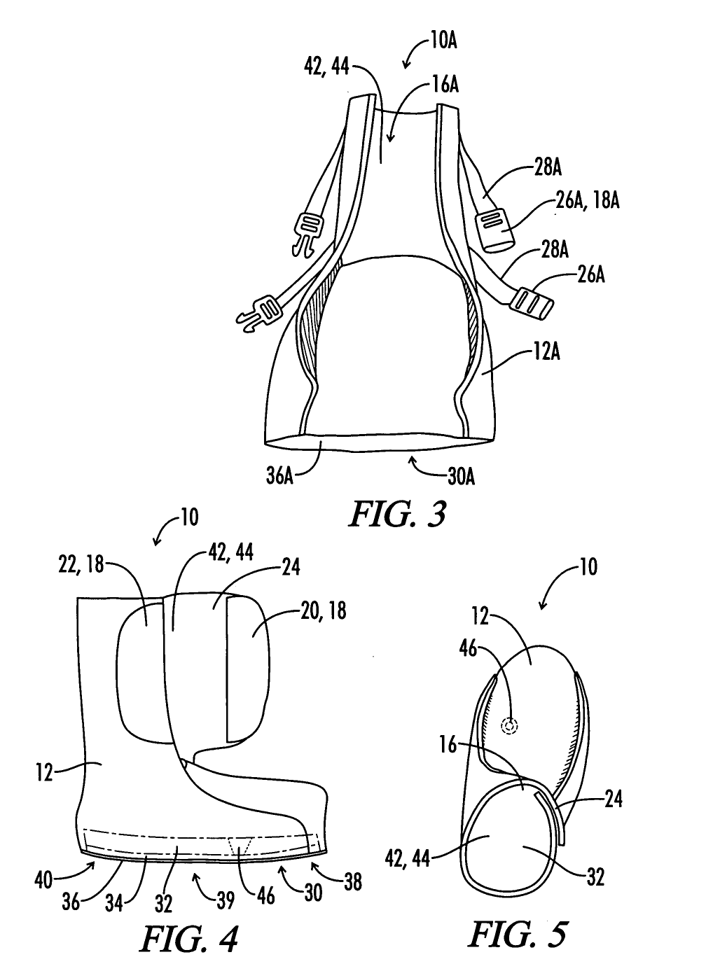

A second embodiment of the invention is depicted in FIG. 3. The components in FIG. 3 are denoted with the suffix A to facilitate distinction between the embodiments. In this second embodiment, the releasable fastener 18A comprises a buckle 26A wherein the buckle 26A is received on the end of a strap 28A. Other types of releasable fasteners 18A could also be used. The strap 28A is positioned to extend across the expansion slot 16A and thereby compress the expansion slot 16A. When the strap embodiment of the boot 10A is used, a flap is not needed for compressing the expansion slot 16A.

Referring again to FIGS. 1 and 2, the upper 12 is comprised of a closed cell polymer. Preferably the polymer is polyethylene, and preferably this polyethylene has been expanded and blown with nitrogen so as to minimize exposure of the patient to irritating chemicals. Some foams use different chemicals for expanding the foam, and the chemicals can be irritating to human skin. Nitrogen is very inert, and does not irritate skin. The upper 12 also includes the releasable fastener 18 as well as other possible additions, but the bulk of the upper 12 is comprised exclusively of the closed cell polymer.

### Sole

The boot 10 also comprises a sole 30, and the sole 30 has three components as best seen in FIGS. 4 and 5. The sole 30 is comprised of an insole 32, a midsole 34, and an outsole 36. The insole 32, midsole 34, and outsole 36 are each connected to the upper, and the insole 32 is also connected to the midsole 34, whereas the midsole 34 is further connected to the outsole 36. Therefore, the insole 32 is connected to the outsole 36 through the midsole 34. Because the insole 32, midsole 34, and outsole 36 are connected to the upper 12, it is possible for these components of the sole 30 to not be attached to each other. The connection to the upper 12 would serve to hold the various parts of the sole 30 in the proper position. It is also possible for the sole 30 to be comprised of just an insole 32 and an outsole 36 without the midsole 34 being present.

The upper 12 covers the edges of the insole 32 and the midsole 34, but the outsole 36 extends across the bottom edge of the upper 12. This form of assembly serves to protect most of the side edge surfaces of the boot 10. The upper 12 protects the edges of the insole 32 and midsole 34, and the outsole 36 protects the bottom edge of the upper 12. Glue or some form of adhesive is used to connect the various parts of the boot 10.

The insole 32 is comprised of a closed cell polymer, preferably a closed cell polyethylene. This insole 32 is the portion of the sole 30 which contacts the foot of the patient when the boot 10 is worn. The midsole 34 is positioned between the insole 32 and the outsole 36 and can be made of a variety of materials to add different characteristics to the boot 10. These materials can include a closed cell polymer or polyethylene, but it can also include a variety of other materials. For example, the midsole 34 can include a shank to stiffen the sole 30. The outsole 36 is intended for ground engagement, and is the part of the sole 30 which contacts the ground. The outsole 36 is comprised of a durable material to withstand the demands from repeated contact with the ground.

The outsole 36 has a rocker shape. This rocker shape allows the patient to roll through the gait, and reduces the shear forces and pressure on the plantar or bottom surface of the foot. This rocker shape involves a raised portion at the toe end 38 of the sole 30 and a raised portion at the heel end 40 of the sole 30 with a lower portion in the middle 39, between the toe 38 and heel 40 portions. The raised portions at the toe 38 and heel 40 are lower closer to the middle 39 of the boot 10, and angle upward as the end of the boot 10 is approached. This provides for a somewhat bow shaped sole 30 with the lowest point being in the middle 39 of the foot and elevated portions at the toe 38 and heel 40 of the sole 30. When the three parts of the sole 30 are connected together and to the upper 12, they form a laminate. Due to the laminations, the sole 30 is a relatively non-flexible surface, and does not bend to a significant extent when the patient walks. This is desirable because it prevents flexion at the ulcer, which facilitates the heeling of the ulcer.

As described, the upper 12 and the insole 32 are comprised of a closed cell polymer, wherein the closed cell polymer is preferably a closed cell polyethylene. The closed cell polymer is preferably expanded with nitrogen as opposed to other expansion agents so as to minimize chemical exposure to the patient. Some foams use different chemicals for blowing or expanding the foam, and the chemicals can be irritating to human skin. Nitrogen is very inert, and does not irritate skin. One version of nitrogen expanded, closed cell polyethylene is sold under the trademark PLASTAZOTE. PLASTAZOTE has been found to be desirable for the treatment of ulcers. This form of closed cell polyethylene is non toxic, non allergenic, latex free, and tends to reduce shear forces where it contacts skin. This form of closed cell polyethylene is also easy to clean so that the boot 10 can be washed and sanitized between uses. Because the primary parts of the boot 10 are comprised of the closed cell polymer, the boot 10 as a whole is mostly closed cell polymer. In fact, the boot 10 is comprised of at least 80% by weight closed cell polymer, wherein this closed cell polymer preferably is closed cell polyethylene.

As a result of the closed cell polymer construction, the boot 10 is very light. In fact, the entire boot 10 weighs less than one pound. Because the sole 30 and the upper 12 are connected, the boot 10 comprises a single article, so there are no parts for the patient to lose. The boot 10, when the ventilation holes 14 are not present, serves to cover the entire foot and a portion of the leg of the patient. This coverage provides protection for the foot which is valuable because the patient often has compromised sensation of the foot. This protection tends to minimize the introduction of foreign bodies or microbes which could irritate the ulcer or perhaps create new injuries for the patient.

Several factors have been described which improve the convenience of the boot 10, and therefore aid in compliance. These factors include the ease of donning and removing the boot 10, due to the large expansion slot 16; the flexibility of the upper, which provides a more natural gait by allowing some flexion of the ankle joint, which in turn tends to decrease chances of a fall; the light weight of the boot 10; and the boot 10 being comprised of a single article to prevent misplacing a piece of the boot. Increased compliance means the patient uses the boot 10 more frequently, which tends to result in better healing of the ulcer.

### Interior Surface

The upper 12 and insole 32 combine to define an interior surface 42 of the boot 10. The interior surface 42 is the portion of the boot 10 which contacts the skin of the patient when the boot 10 is worn. The interior surface 42 is custom molded to one particular person. Preferably this is done through the use of a cast of the foot and part of the leg of the particular individual who will use the boot 10. A cast is made of the foot and leg of the individual, and this cast is sent to a location where the boot 10 is molded. The ulceration site is marked on the cast so the interior surface 42 can be formed to accommodate the ulcer of the person. Because a cast is used, it is not necessary for the patient to travel to the site where the boot 10 is molded. This increases the convenience and reduces the overall cost to the patient for the use of the boot 10.

The boot 10 is custom molded using vacuum and heat. The mold or cast is placed inside the boot 10 and a vacuum is drawn between the cast and the interior surface 42 of the boot 10. This vacuum serves to draw the boot 10 around the cast. At the same time, the boot 10 and cast are heated. The heat softens the material in the boot 10, allowing the interior surface 42 to change and mold to the dimensions and contours of the foot and leg of the patient. When the boot 10 cools, the material in the boot 10 retains the shape from the molding process.

The heat and vacuum molding provides a boot 10 with a total contact casting of the plantar surface of the foot, the upper surface of the foot, and part of the lower leg. A total contact casting involves contouring the interior surface 42 to substantially match all the contours of the reference surface, such as the foot. This serves to spread the weight over the entire surface of the foot, because places like the arch that normally don't bear any significant weight are supported, and become weight bearing surfaces. Because of this molding, the interior surface 42 has dimensions 44 or contours 44 which substantially define the foot and part of the leg of the one particular individual for whom the boot 10 is intended. Extra space can be formed into the interior surface 42 to allow for movement of toes and similar needs.

Custom molding creates a boot 10 which fits the particular individual. Because of this, the boot 10 does not slip on the foot or leg of the patient. This minimizes shear forces and ensures that the boot 10 stays in the proper location. The interior surface 42 also includes a cut out 46 which is positioned to surround the ulcer of the patient. The cut out 46 is formed based on the marking on the cast which indicated the location of the ulcer. Because the boot 10 is custom molded to the foot and leg, the cut out 46 remains properly positioned surrounding the ulcer of the patient. This is true if the ulcer is on the plantar surface of the foot, on top of the foot, or on the leg. If the patient has more than one ulcer, the interior surface 42 can include more than one cut out 46. When the ulcer is positioned where cut out 46 surrounds the ulcer, there is no weight placed on the ulcer because there is no surface for the ulcer to press against. Additionally, the boot can undergo further adjustments for when a subsequent ulcer develops during a specific healing period.

### Method

The current invention also includes a method of protecting an ulcer. This method comprises providing a boot 10 having an upper 12 and an insole 32, wherein the upper 12 is dimensioned to extend over at least a part of the leg of the patient. The boot 10 is comprised of at least 80% by weight closed cell polymer, wherein the polymer is preferably polyethylene. The upper 12 includes an expansion slot 16 to facilitate donning and removing of the boot 10, and the boot 10 further comprises an outsole 36 having a rocker shape to facilitate walking.

An interior surface 42 of the boot 10 is molded such that the interior surface 42 form fits the foot and part of the leg of the one particular individual who will use the boot 10. The method of molding the boot 10 to the patient involves forming a cast of the foot and part of the leg of the particular individual and vacuum and heat molding the interior surface 42 of the boot 10 to form fit the cast and thus the patient. The boot 10 should be remolded to the foot and part of the leg of the particular individual if and when the dimensions of the foot and leg of the particular individual change, A cut out 46 is then created in the interior surface 42 of the boot 10 wherein the cut out is positioned to surround the ulcer of the patient. The boot 10 is then worn by the patient, and should be washed and sanitized between uses. The boot 10 can be disinfected when needed.

Thus, although there have been described particular embodiments of the present invention of a new and useful BOOT FOR ULCER TREATMENT, it is not intended that such references be construed as limitations upon the scope of this invention except as set forth in the following claims.

The disclosures of all cited patents and publications referred to in this application are incorporated therein by reference.

What is claimed is:

1. A method of protecting an ulcer comprising:
(a) providing a boot having an upper and an insole wherein the upper is dimensioned to extend over at least a part of a leg;
(b) molding an interior surface of the boot such that the interior surface form fits a foot and part of the leg of one particular individual; and
(c) creating a cutout in the interior surface of the boot, the cutout positioned to surround the ulcer.

2. The method of claim 1 wherein the boot is comprised of at least 80 percent by weight closed cell polyethylene.

3. The method of claim 1 wherein step (b) further comprises:
forming a cast of the foot and part of the leg of the particular individual; and
vacuum and heat molding the interior surface of the boot to form fit the cast.

4. The method of claim 1 wherein step (a) further comprises providing an expansion slot in the upper to facilitate the donning and removal of the boot.

5. The method of claim 1 wherein step (a) further comprises providing a rocker shaped outsole to facilitate walking.

6. The method of claim 1 further comprising washing and sanitizing the boot between uses.

7. The method of claim 1 further comprising adjusting the boot to the foot and part of the leg of the particular individual for the development of a subsequent ulcer.

8. A boot to aid in the treatment of lower extremity ulcers comprising:
an upper dimensioned to extend up at least part of a leg, wherein the upper is flexible;
an insole connected to the upper;
an outsole connected to the upper, the outsole for ground engagement, wherein the boot comprises a single article; and
an interior surface defined by the upper and the insole wherein the interior surface includes a cutout positioned to surround the ulcer.

9. The boot of claim 8 wherein the boot includes at least 80% by weight closed cell polymer.

10. The boot of claim 9 wherein the closed cell polymer is closed cell polyethylene.

11. The boot of claim 9 wherein the boot weighs less than 1 pound.

12. The boot of claim 8 wherein the outsole is rocker shaped.

13. The boot of claim 8 wherein the upper includes an expansion slot, and wherein the boot includes a releasable fastener connected to the upper, the releasable fastener positioned to compress the expansion slot.

14. The boot of claim 8 wherein the interior surface dimensions substantially define a foot and part of a leg of one particular individual.

15. The boot of claim 8 wherein the upper includes ventilation holes.

16. A boot to aid in the treatment of lower extremity ulcers comprising:
an upper including an expansion slot and a flap, the flap defined adjacent the expansion slot such that the flap overlaps the expansion slot, wherein the flap is releasably secured to the upper;
an insole connected to the upper; and
an interior surface defined by the insole and the upper, wherein the interior surface includes a cutout positioned to surround the ulcer.

17. The boot of claim 16 wherein the boot is comprised of at least 80 percent by weight closed cell polymer.

18. The boot of claim 17 wherein the polymer is polyethylene.

19. The boot of claim 16 wherein a contour of the interior surface substantially defines a foot and part of a leg of one particular individual.

20. The boot of claim 16 wherein the boot weighs less than 1 pound.

21. The boot of claim 16 further comprising a rocker shaped outsole connected to the upper.

22. The boot of claim 16 wherein the boot comprises a single article.
